(19) United States
(12) Patent Application Publication
(10) Pub. No.: US 2018/0014380 A1
(43) Pub. Date: Jan. 11, 2018

(54) Battery Powered Circuit For Ignition And Operation Of A UV Lamp

(71) Applicant: LUMIFRESH LLC, Beverly Hills, CA (US)

(72) Inventors: Radoslaw Marian Kornicki, Ames, IA (US); Radoslaw Przybysz, Celestynow (PL)

(21) Appl. No.: 15/642,040

(22) Filed: Jul. 5, 2017

Related U.S. Application Data
(60) Provisional application No. 62/358,472, filed on Jul. 5, 2016.

(51) Int. Cl. H05B 37/02; B08B 7/00; A61L 2/10

(52) U.S. Cl. CPC H05B 37/0227; A61L 2/10; B08B 7/0057

(57) ABSTRACT

Typical prior art circuits for ignition and operation of UV lamps do not optimize power usage through the whole cycle of ignition and ongoing operation. Typically, the higher power required to initially ignite the UV lamp is not diminished once the UV lamp is burning brightly and at a desired temperature. The circuit of the present invention utilizes a sensor to monitor the light emitted from the UV lamp and a sensor to measure the temperature of the device containing the UV lamp as voltage is applied to it. Once the constant light and desired temperature are achieved, the voltage boost converter is latched away and constant lower voltage is provided to the UV lamp from that point forward, thus optimizing the energy consumption or the energy used for a battery powered device incorporating a UV lamp.

### CROSS-REFERENCES TO RELATED APPLICATIONS

This application claims the benefit of U.S. Provisional Application Serial No. 62/358,472 filed on Jul. 5, 2016 titled “BATTERY POWERED CIRCUIT FOR IGNITION AND OPERATION OF A UV LAMP” which is incorporated herein by reference in its entirety for all that is taught and disclosed therein.

### BACKGROUND

This disclosure pertains to UV lamps and ignition and control circuits, and more particularly, to battery operated devices that incorporate a UV lamp and an ignition and control circuit.

### SUMMARY

This Summary is provided to introduce in a simplified form a selection of concepts that are further described below in the Detailed Description. This Summary is not intended to identify key features or essential features of the claimed subject matter, nor is it intended to be used to limit the scope of the claimed subject matter.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or class of elements, such as X1-Xm, Y1-Yn, and Z1-Zo, the phrase is intended to refer to a single element selected from X, Y, and Z, a combination of elements selected from the same class (e.g., X1 and X2) as well as a combination of elements selected from two or more classes (e.g., Y1 and Z3 ).

It is to be noted that the term “a entity” or “an entity” refers to one or more of that entity. As such, the terms “a” (or “an”), “one or more,” and “at least one” can be used interchangeably herein. It is also to be noted that the terms “comprising,” “including,” and “having” can be used interchangeably.

Unless the meaning is clearly to the contrary, all ranges set forth herein are deemed to be inclusive of the endpoints.

The detailed description below describes an ignition and control circuit for a UV lamp within a battery powered device. The solution described below enables a sensor to monitor the light emitted from the UV lamp as voltage is applied to it. Once the constant light and desired temperature are achieved, the voltage boost converter is latched away and constant lower voltage is provided to the UV lamp from that point forward, thus optimizing the energy consumption or the energy used for a battery powered device incorporating a UV lamp.

### BRIEF DESCRIPTION OF THE DRAWINGS

FIG. 1 shows a portable case for oral appliances in a closed position that uses UV light and optionally an ozone generator to kill bacteria and odor utilizing the circuit of the present invention.

FIG. 2 shows a portable case for oral appliances in an open position that uses UV light and optionally an ozone generator to kill bacteria and odor utilizing the circuit of the present invention.

FIG. 3 shows a schematic/block diagram of an embodiment of a circuit for controlling a UV lamp of the prior art.

FIG. 4 shows a schematic/block diagram of an embodiment of a circuit for the ignition and operation of a UV lamp of the present invention.

FIG. 5 shows an exploded view of an oral appliance that uses UV light to kill bacteria and odor utilizing the circuit of the present invention.

FIG. 6 shows a flow chart of the method of operation of the battery-powered circuit for UV lamp ignition and operation of the present invention.

FIG. 7 shows a flow chart of a safety operating method that runs continuously in the background when the device is powered on of the present invention.

To assist in the understanding of the present disclosure the following list of components and associated numbering found in the drawings is provided herein:

Table of Components

| Component | # |
|---|---|
| oral appliance cleansing device | 100 |
| top half | 102 |
| bottom half | 104 |
| battery | 106 |
| main printed circuit board | 108 |
| lower inner shell | 110 |
| lower UV lamp | 112 |
| lower UV lens | 114 |
| upper UV lens | 116 |
| upper UV lamp | 118 |
| upper inner shell | 120 |
| user interface printed circuit board | 122 |
| LEDs | 124 |
| touch sensing pad | 126 |
| cavity | 128 |
| circuit | 300 |
| battery-powered circuit | 400 |
| method | 600 |
| method | 700 |
| battery power supply | BAT |
| charge control | CC |
| control unit | CU |
| divider one | D1 |
| divider two | D2 |
| energy bank | EB |
| housing | H |
| temperature sensor | TS |
| input signaling element | ISE |
| boost coil | L |
| light sensor | LS |
| output | M1 |
| input | M2 |
| output | M3 |
| input | M4 |
| input | M5 |
| input | M6 |
| input | M7 |
| input | M8 |
| input | M9 |
| microprocessor | MCU |
| measuring electrode | ME |
| proximity detector | PD |
| power input | PI |
| power steering bridge | PSB |
| shorting key terminal | SKT |
| step-up transformer | SUT |
| type coupler | TC |
| UV lamp | UV |
| voltage boost converter | VBC |

### DETAILED DESCRIPTION

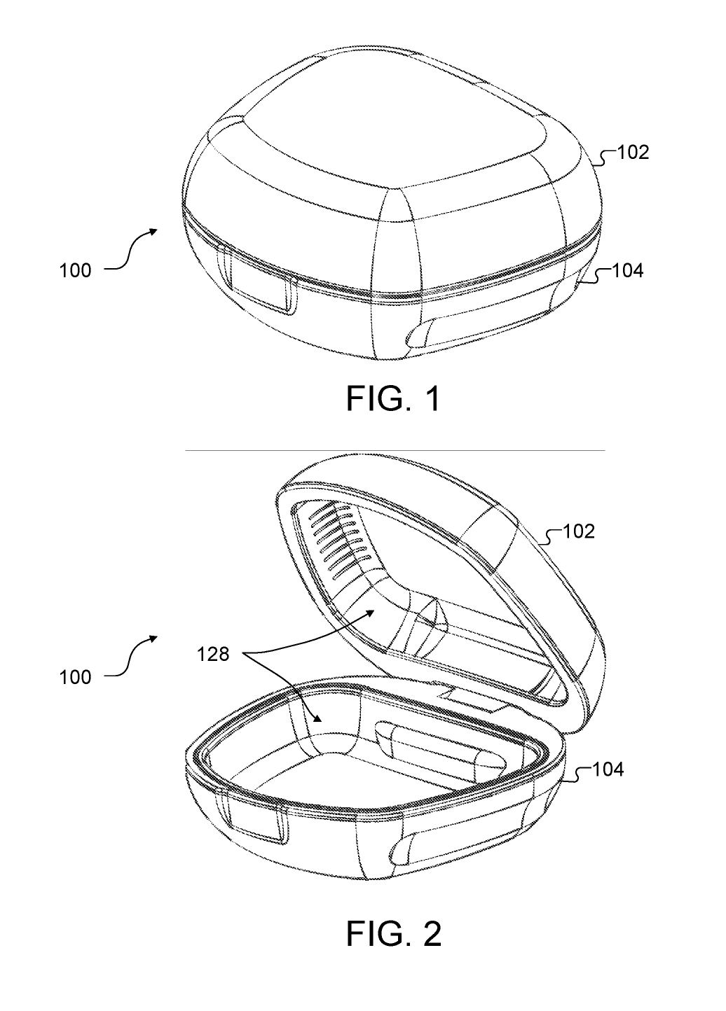

Referring now to the Figures, in which like reference numerals refer to structurally and/or functionally similar elements thereof, FIGS. 1 and 2 show a portable case for oral appliances in a closed position and an open position that uses UV light and optionally an ozone generator to kill bacteria and odor utilizing the circuit of the present invention. Referring now to FIGS. 1 and 2, the oral appliance cleansing device 100, shown in a closed position in FIG. 1, combines the antibacterial use of a UV light and optionally an ozone generator together in a single unit. Oral appliance cleansing device 100 is clean, compact, and easy to use. FIG. 2 shows the portable case for oral appliances in an open position. A user places his or her oral appliance (not shown) inside oral appliance cleansing device 100 and closes a top half 102 against a bottom half 104. In one embodiment, closing the top half 102 against the bottom half 104 activates the circuit that turns on the UV light and the optional ozone generator. In another embodiment, a mechanical switch (not shown), or a touch sensor (not shown), on the outside of oral appliance cleansing device 100 activates the circuit.

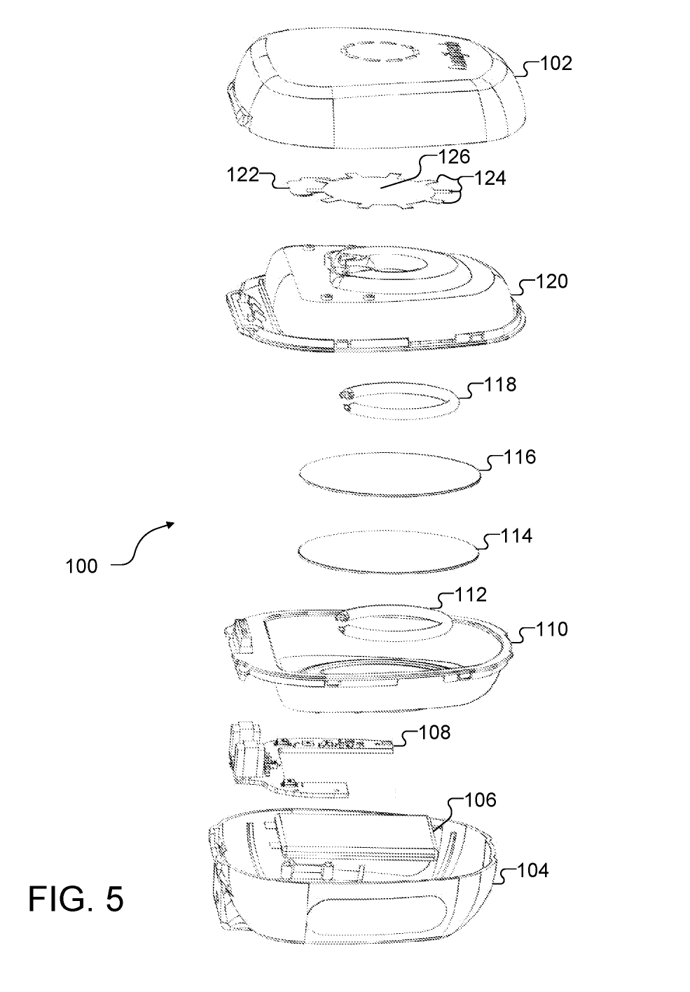

FIG. 5 shows an exploded view of an oral appliances that uses UV light to kill bacteria and odor utilizing the circuit of the present invention. Referring now to FIG. 5, a battery 106 is inserted in a lower portion of bottom half 104. Main printed circuit board 108 connects to and derives power from battery 106. Lower inner shell 110 protects main printed circuit board 108 and battery 106, and forms the bottom portion of a cavity 128 within oral appliance cleansing device 100 that will receive an oral appliance (not shown). Lower UV lamp 112 is covered by lower UV lens 114 and both are secured to the lower portion of lower inner shell 110.

User interface printed circuit board 122 secured between top half 102 and upper inner shell 120 and connects to main printed circuit board 108 (connections not shown). The user interface printed circuit board 122 houses several LEDs 124 to inform a user about the device status and is the primary function of user interface printed circuit board 122. In addition, located on the user interface printed circuit board 122 is a touch sensing pad 126 made of copper that changes capacitance when a touch is made, similar to the way touch screens work. User interface printed circuit board 122 carries the signals that control the several LEDs 124 and senses touch input. The main printed circuit board 108 is where the lower UV lamp 112 and upper UV lamp 118 are controlled by a signal that is acquired from the user interface printed circuit board 122. Microprocessor MCU is located on main printed circuit board 108 and is where the logic for operation of oral appliance cleansing device 100 resides. User interface printed circuit board 122 houses a tiny microprocessor that essentially collects information about touch events and generates the commands to activate the several LEDs 124.

Upper inner shell 120 forms the top portion of the cavity 128 within oral appliance cleansing device 100 that will receive an oral appliance (not shown). Upper UV lamp 118 is covered by upper UV lens 116 and both are secured to the upper portion of upper inner shell 120.

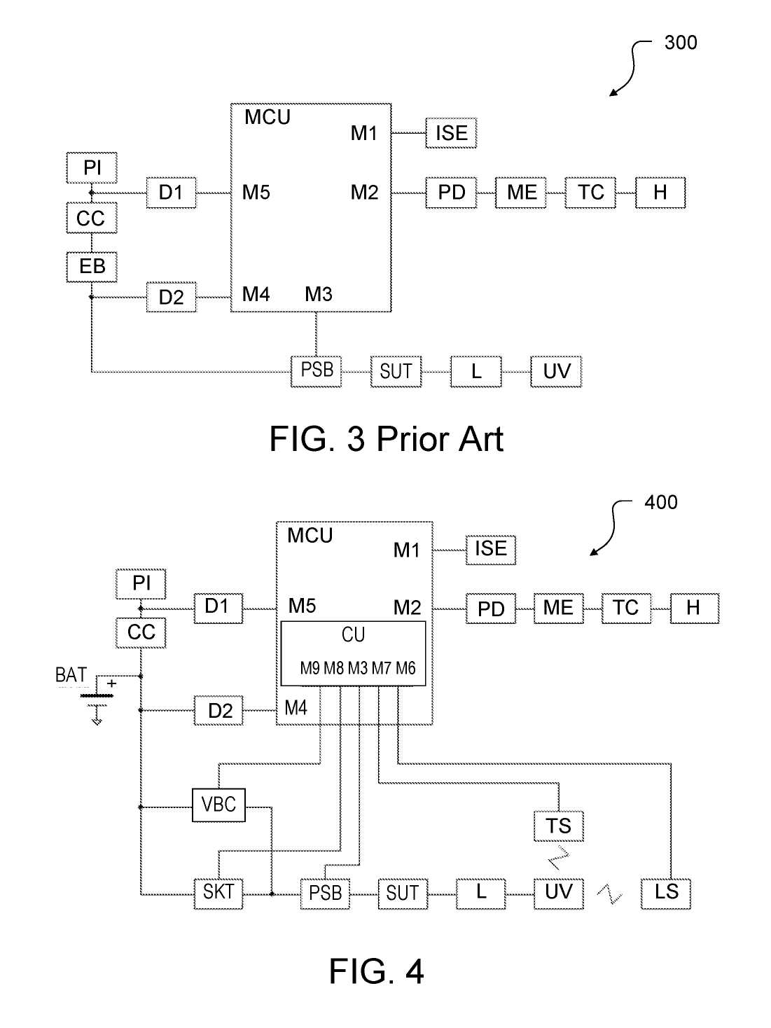

FIG. 3 shows a schematic/block diagram of an embodiment of a circuit 300 for controlling a UV lamp of the prior art. Referring now to FIG. 3, Output M1 of the microprocessor unit MCU is connected to the input signaling element ISE which provides the means for command input, such as through touch sensing pad 126, or in another embodiment, input buttons. Input M2 to microprocessor unit MCU is connected to the output of proximity detector PD, whose input is connected to the specially shaped measuring electrode ME, which through a special type coupler TC is connected to the housing H.

The output of power input terminal PI is connected to the input of divider one D1, the output of which is connected to an input M5 of the microprocessor unit MCU.

Output of power input terminal PI is connected to the input of the charge control CC, the output of which is connected to the input of the energy bank EB, the output of which is connected to the input of divider two D2, the output of which is connected to the input M4 of microprocessor unit MCU. The output of the energy bank EB is connected with input of the power steering bridge PSB, which output is connected to the step-up transformer SUT, the output of which is connected to the input of the boost coil L which output is connected to UV lamp UV. The control input of the power steering bridge PSB is connected to the output M3 of the microprocessor unit MCU.

The method of operation of this prior art circuit 300 is as follows. A typical UV lamp requires a relatively high voltage to actually start the ignition of the lamp. The circuit uses a resonance effect of voltage resulting from a series connection of coil L and UV lamp UV. The microprocessor unit MCU controls the power steering bridge PSB with a square wave, which uses a step-up transformer SUT to generate high voltage on the boost coil L.

FIG. 4 shows a block diagram of an embodiment of a battery-powered circuit 400 for controlling a UV lamp of the present invention. Referring now to FIG. 4, the description above in relation to FIG. 3 for the elements MCU, PI, D1, D2, CC, ISE, PD, ME, TC, H, M1, M2, M3, M4, and M5 apply to the similarly labeled elements in FIG. 4, and are not repeated here.

Battery-powered circuit 400 for ignition and operation of UV lamp UV consists of battery power supply BAT. The positive terminal is input to a shorting key terminal SKT, which is connected along with the input to voltage boost converter VBC. The energy bank EB shown in FIG. 3 is comparable to battery power supply BAT. Output of the shorting key terminal SKT and the output of voltage boost converter VBC are connected together and then connected to the input of power steering bridge PSB. Output of the power steering bridge PSB is connected to the input of step-up transformer SUT, the output of which is connected to the input of boost coil L, the output of which is connected to UV lamp UV. The light emitted by the UV lamp UV is received by light sensor LS, the output of which is connected to an input M6 to the control unit CU. The heat emitted by the UV lamp UV is received by temperature sensor TS, the output of which is connected to an input M7 to the control unit CU. In an alternative embodiment, temperature sensor TS is an integrated temperature sensor that is built into microprocessor MCU. The temperature of the whole device can thus be sensed allowing for the inclusion of sensing alarm features such as battery overheating. One thing is that it does not need to be physically connected to the processor—it can be one that is built in. The control unit CU is connected to the input M3 of the power steering bridge PSB, the input M9 of the voltage boost converter VBC, and the input M8 of shorting key terminal SKT.

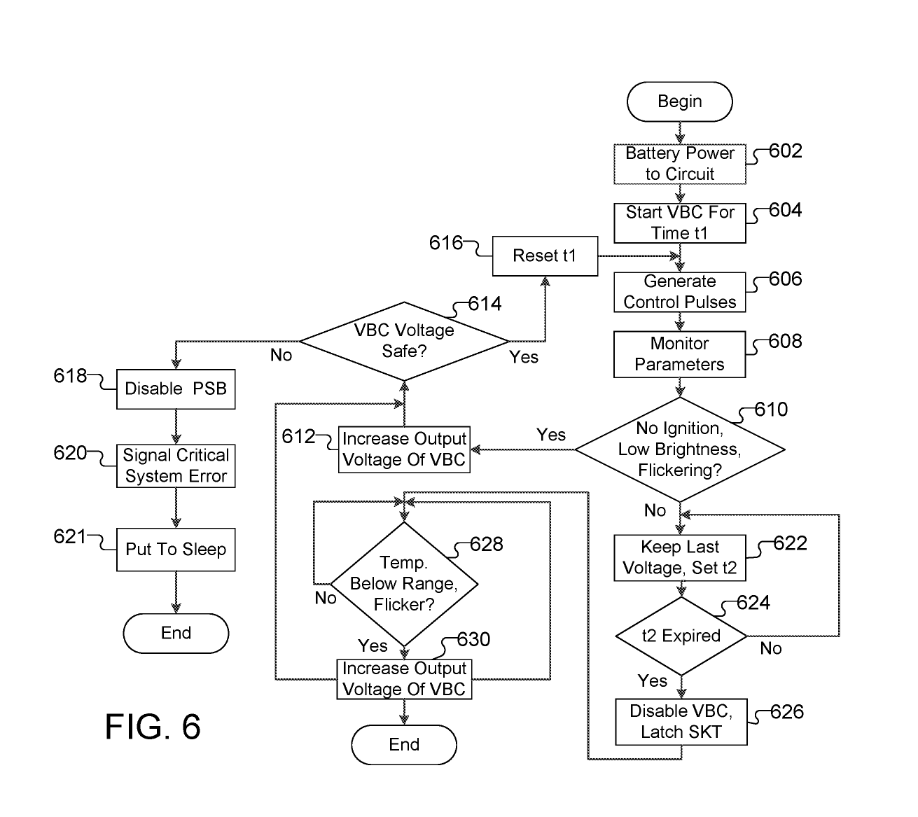

FIG. 6 shows a flow chart of the method of operation of the battery-powered circuit for UV lamp illumination of the present invention. Referring now to FIG. 6, method 600 begins in block 602 by connecting and switching on battery power supply BAT to the circuit 400. In block 604 control unit CU starts the voltage boost converter VBC for a time t1, typically a few seconds. In block 606 control unit CU starts the generation of control pulses on the input of power steering bridge PSB, monitoring at the same time in block 608 the brightness level and/or flickering of UV lamp UV. In decision block 610, if there is no ignition or low brightness level of the UV lamp UV, or UV lamp UV flickers, then in block 612 control unit CU increases the VBC. Block 614 determines whether the new operating voltage is within safe operational limits. If yes, then block 616 resets time t1 in control unit CU and control returns to block 606.

If block 614 determines that parameters fall outside of safe operating limits, then block 618 disables control pulses on input of power steering bridge PSB and block 620 signals a critical system error. In block 621 control unit CU puts the device to sleep and the process ends.

When control returns to block 606 the cycle is continued until the brightness level and lack of flickering is achieved. Brightness or lack thereof, and flickering or lack thereof, are measured with a photo diode of the light sensor LS, where the current from the light sensor LS is proportional to light intensity. In one embodiment, a predetermined value for the current indicates satisfactory brightness. Flickering is defined as an absence of light for a certain duration of time. In one embodiment flickering is determined by several light on/light off events within a span of a second. Flickering in another embodiment is determined by a lack of signal for a duration of 0.2 seconds between received signals. In other embodiments, the predetermined value for the current and the absence of light may be adjusted for specific applications.

In decision block 610, when the UV lamp UV reaches the expected level of brightness and it does not flicker, in block 622 the control unit VBC keeps the set parameters (the last voltage level used) for time t2, causing the UV lamp UV to heat up. The initial parameters for a particular device are determined experimentally and are pre-programmed into the microprocessor unit MCU for the device. Time t2 varies based on multiple parameters, such as the ambient temperature and by manufacturing tolerances of components from device to device. Time t2 will thus vary from device-to-device, and may range from several seconds to several minutes. If decision block 624 determines that the time t2 has not expired, control returns to block 622. If decision block 624 determines that the time t2 has expired and stable lamp ignition has been achieved, then in block 626 the control unit CU disables the voltage boost converter VBC and latches the shorting key terminal SKT at the same time and constant lower voltage is provided to UV lamp UV.

Decision block 628 determines if the temperature drops below the predetermined temperature stabilization range, or if flicker is detected. If either is yes, then in block 630 the voltage is increased and control returns to decision block 628. If the determination in decision block 628 is no, control returns to this block for continued monitoring of the temperature and detection of flicker. The method ends when the circuit is powered off.

Through this method, the UV lamp UV can be illuminated at any ambient temperature (the colder or hotter the ambient temperature is will affect the time to obtain the preheat temperature) and partial discharge of the battery. The battery power supply BAT will output lower voltage at a given load as the device operates. This lower voltage will impact input voltage to the voltage boost converter VBC and affect output voltage.

Battery-powered circuit 400 takes advantage of actually closing a loop with the UV lamp UV and decreasing the voltage in the UV lamp UV once the UV lamp UV achieves the successful glow (no flickering) and once the temperature stabilizes. The control algorithm for powering the UV lamp UV monitors output of the UV lamp UV and controls input to the voltage boost converter VBC, referred to as a closed control loop. Temperature stabilization may vary from device-to-device and lamp-to-lamp. In one embodiment, temperature stabilization was achieved within +/− three degrees Celsius. In other embodiments, temperature stabilization may be less than or greater this range.

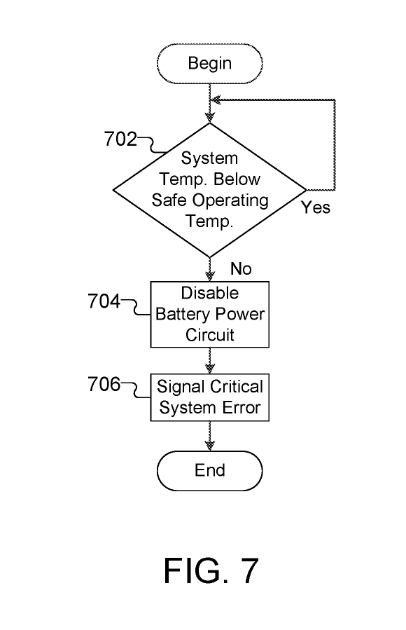

FIG. 7 shows a flow chart of a safety operating method that runs continuously in the background when the device is powered on. Referring now to FIG. 7, method 700 begins in decision block 702 which determines if the temperature of the device stays below a safe operating temperature. If yes, control returns to this decision block for further monitoring to make sure the temperature does not rise to an unsafe level. If no, in block 704 the battery power circuit is disabled, and block 706 signals a critical system error and the method ends.

In the circuit shown in FIG. 3 the energy balance in the UV lamp UV is not optimized throughout the whole cycle. Energy is pumped the into UV lamp UV whether it needs it or not. Battery-powered circuit 400 shown in FIG. 4 reduces the voltage after the UV lamp UV attains the right glow and temperature by means of the shorting key terminal SKT. Once the UV lamp UV is turned on and is successfully glowing, and once the temperature of the UV lamp UV actually reaches a little bit higher temperature, higher power is simply not needed (as is present in circuit 300) to sustain the illumination of UV lamp UV. Battery-powered circuit 400 shorts the connection and bypasses the voltage boost converter VBC to sustain UV lamp UV. Light sensor LS and temperature sensor TS monitor what is happening to the light and heat emitted by oral appliance cleansing device 100 as voltage is applied to it. Control unit CU makes decisions based on what light sensor LS and temperature sensor TS are picking up, and once the constant light and desired stabilized temperature are achieved, the voltage boost converter VBC is latched away and constant lower voltage is provided to UV lamp UV from that point forward. If the temperature of the UV lamp UV drops outside of the predetermined temperature stabilization range, the voltage is increased. Voltage will also be increased if flicker is detected, and if the temperature falls below the predetermined temperature stabilization range. Control unit CU resides in the microprocessor unit MCU as shown in FIG. 4. Battery-powered circuit 400 monitors what is happening to the light emitted by UV lamp UV when voltage is applied to it. Battery-powered circuit 400 closes the loop and optimizes the energy consumption or the energy used for a battery powered device. Although battery-powered circuit 400 is shown in a specific application of oral appliance cleansing device 100, the principles taught by battery-powered circuit 400 could work with any UV light application, scaled up or down based on the size of the lamp.

Having described the present invention, it will be understood by those skilled in the art that many changes in construction and circuitry and widely differing embodiments and applications of the invention will suggest themselves without departing from the scope of the present invention.

What is claimed is:

1. A method for ignition and operation of a UV lamp contained in a device, the method comprising the steps of:
(a) applying power to a circuit;
(b) starting a voltage boost converter for a period of time t1;
(c) generating control pulses on a power steering bridge;
(d) monitoring an ignition, a brightness level, and/or a flickering of a UV lamp;
(e) determining if the UV lamp does not have the ignition, does not have the brightness level required, and/or does have the flickering:
(e1) if yes, increasing an output voltage of the voltage boost converter;
resetting the period of time t1; and
repeating steps (c), (d), and (e);
(e2) if no, keeping the output voltage of the voltage boost converter to the output voltage last used for a period of time t2; and
(f) determining if the period of time t2 has expired, and/or a temperature of the device has been achieved that falls within a predetermined temperature stabilization range:
(f1) if no, repeating step (e2);
(f2) if yes, disabling the voltage boost converter;
latching a shorting key terminal; and
providing a constant lower voltage to the UV lamp.

2. The method for ignition and operation of a UV lamp according to claim 1 further comprising the step of:
(g) determining if the temperature of the device falls below the predetermined temperature stabilization range, and/or the flickering of the UV lamp occurs;
(g1) if yes, increasing the constant lower voltage to the UV lamp; and
repeating step (g); and
(g2) if no, repeating step (g).

3. The method for ignition and operation of a UV lamp according to claim 2 further comprising the steps of:
performing steps (b) through (g2) with a control unit within a microprocessor.

4. The method for ignition and operation of a UV lamp according to claim 3 further comprising the step of:
pre-programming the period of time t1 and the period of time t2 into the microprocessor.

5. The method for ignition and operation of a UV lamp according to claim 3 wherein step (f) further comprises the step of:
measuring the temperature of the device with a temperature sensor that sends signals representative of the temperature of the device to the control unit.

6. The method for ignition and operation of a UV lamp according to claim 1 wherein step (a) further comprises the step of:
switching on a battery to supply power to the circuit.

7. The method for ignition and operation of a UV lamp according to claim 1 wherein step (d) further comprises the step of:
measuring the brightness level with a photo diode in a light sensor, wherein a current from the light sensor is proportional to a light intensity coming from the UV lamp, and a predetermined value for the current from the light sensor indicates that the brightness level is satisfactory.

8. A circuit for ignition and operation of a UV lamp contained in a device, the circuit comprising:
a control unit within a microprocessor;
a voltage boost converter started by the control unit that supplies a voltage to a UV lamp;
a power steering bridge that receives control pulses from the control unit;
a light sensor for monitoring the UV lamp that sends a current proportional to a light intensity from the UV lamp to the control unit;
a temperature sensor for measuring a temperature of the device that sends signals representative of the temperature to the control unit; and
a shorting key terminal that the control unit latches, and the control unit disables the voltage boost converter when the temperature of the device falls within a predetermined temperature stabilization range and achieves a brightness level, and the control unit supplies a constant lower voltage to the UV lamp.

9. The circuit for ignition and operation of a UV lamp according to claim 8 further comprising:
a step-up transformer that receives the output of the power steering bridge; and
a boost coil that receives high voltage generated by the step-up transformer whose output is connected to the UV lamp.

10. The circuit for ignition and operation of a UV lamp according to claim 8 further comprising:
an input signaling element that receives a first output from the microprocessor.

11. The circuit for ignition and operation of a UV lamp according to claim 8 further comprising:
a proximity detector whose output is connected to the microprocessor;
a specially shaped measuring electrode whose output is connected to the proximity detector;
a special type coupler which connects to the specially shaped measuring electrode; and
a housing connected to the special type coupler.

12. The circuit for ignition and operation of a UV lamp according to claim 8 further comprising:
a battery whose positive terminal is the input to the shorting key terminal.

13. The circuit for ignition and operation of a UV lamp according to claim 12 further comprising:
a power input terminal;
a charge control whose input is received from the power input terminal, and whose output is connected to the input of the battery;
a first divider whose input is received from the power input terminal and whose output is connected to the microprocessor; and
a second divider whose input is received from the battery and whose output is connected to the microprocessor.

14. An oral appliance cleansing device comprising:
a top half connectable to a bottom half with a cavity contained therein for receiving an oral appliance;
an upper UV lamp located in the top half and a lower UV lamp located in the bottom half;
a circuit controlled by a main printed circuit board located in the bottom half;
a battery located in the lower half for providing a voltage to the circuit;
a voltage boost converter in the circuit that supplies the voltage to the upper UV lamp and the lower UV lamp;
a light sensor in the circuit for monitoring the light emitted from the upper UV lamp and the lower UV lamp as the voltage is applied;
a temperature sensor in the circuit for measuring a temperature of the oral appliance cleansing device; and
a shorting key terminal in the circuit that latches away the voltage boost converter when a constant light and a desired temperature are achieved, and a lower voltage is then supplied to the upper UV lamp and the lower UV lamp.

15. The oral appliance cleansing device according to claim 14 further comprising:
a user interface printed circuit board connected to the main printed circuit board;
a plurality of LEDs housed in the user interface printed circuit board; and
a touch sensing pad located on the printed circuit board, wherein the plurality of LEDs output light to indicate a status of the oral appliance cleansing device, and the touch sensing pad captures touch events.
